(19) United States
(12) Patent Application Publication
(10) Pub. No.: US 2023/0368917 A1
(43) Pub. Date: Nov. 16, 2023

(54) ULTRASOUND TIME-SERIES DATA PROCESSING DEVICE AND ULTRASOUND TIME-SERIES DATA PROCESSING PROGRAM

(71) Applicant: FUJIFILM Healthcare Corporation, Kashiwa-shi (JP)

(72) Inventors: Eiji Kasahara, Chiba (JP); Tomoaki Chono, Chiba (JP); Katsunori Asafusa, Chiba (JP)

(21) Appl. No.: 18/138,494

(22) Filed: Apr. 24, 2023

(30) Foreign Application Priority Data
May 16, 2022 (JP) 2022-080336

(51) Int. Cl. G16H 50/20

(52) U.S. Cl. CPC G16H 50/20

(57) ABSTRACT

In a Doppler mode, a Doppler processing unit generates Doppler data as target time-series data on the basis of a received beam data sequence from a reception unit. In an M mode, a beam data processing unit generates a received beam data sequence subjected to various types of signal processing as the target time-series data. A disease prediction unit inputs target time-series data to a disease prediction learner trained to predict and output a disease indicated by the time-series data on the basis of the input time-series data, and predicts the disease indicated by the target time-series data on the basis of output data of the disease prediction learner for the target time-series data. A display control unit notifies a user of the result of the prediction by the disease prediction unit.

### CROSS REFERENCE TO RELATED APPLICATION

This application claims priority to Japanese Patent Application No. 2022-080336 filed on May 16, 2022, which is incorporated herein by reference in its entirety including the specification, claims, drawings, and abstract.

### TECHNICAL FIELD

The present specification discloses an ultrasound time-series data processing device and an ultrasound time-series data processing program.

### BACKGROUND

Conventionally, ultrasound waves are repeatedly transmitted and received a plurality of times to and from the same position (the same direction as viewed from an ultrasound probe) in a subject, and time-series data in the form of a time-series received beam data sequence obtained by the repetition are converted into an image or analyzed.

Examples of the image into which the time-series data are converted include an M-mode image in which the horizontal axis indicates time and the vertical axis indicates a depth and in which the state of movement of tissue in the depth direction is indicated by a luminance line extending in the time axis direction, or a Doppler waveform image in which the position of an examined region or the velocity of blood flowing in the examined region is calculated based on the difference between the frequency of the transmitted ultrasound wave and the frequency of the received ultrasound wave and in which the horizontal axis indicates time and the vertical axis indicates the velocity.

WO 2012/008173 A discloses, as a method for analyzing time-series data, a method for determining a vascular disease, particularly arteriosclerosis, vascular stenosis, or an aneurysm, with high accuracy in a non-invasive manner, the method including: receiving a reflected echo whose frequency has changed to f₀ by transmitting an ultrasound wave (frequency f) to a blood vessel wall of a beating subject; performing wavelet transformation on the reflected echo to acquire a wavelet spectrum; performing mode decomposition on the wavelet spectrum to acquire a spectrum for each mode; acquiring a waveform for each mode on a time axis by wavelet inverse transformation; calculating a norm value for each mode; and comparing the norm values with a norm distribution obtained from a normal individual to determine the presence or absence of a vascular disease or the morbidity of a specific vascular disease.

As described above, conventionally, a disease has been determined by analyzing time-series data, but there has been a problem of an increased amount of calculation for the determination. For example, in WO 2012/008173 A described above, it is necessary to perform various processes of acquiring a wavelet spectrum by wavelet transformation, performing mode decomposition on the wavelet spectrum to acquire a spectrum for each mode, acquiring a waveform for each mode on a time axis by wavelet inverse transformation, calculating a norm value for each mode, and comparing the norm values with a norm distribution obtained from a normal individual.

An object of an ultrasound time-series data processing device disclosed in the present specification is to reduce the amount of calculation for predicting a disease indicated by time-series data based on the time-series data obtained by repeatedly transmitting and receiving ultrasound waves a plurality of times to and from the same position in a subject.

### SUMMARY OF THE INVENTION

An ultrasound time-series data processing device disclosed in the present specification includes: a disease prediction unit that inputs target time-series data to a disease prediction learner and predicts a disease indicated by the target time-series data on the basis of an output of the disease prediction learner in response to the input, the disease prediction learner being trained to predict and output the disease indicated by the time-series data on the basis of the input time-series data, the target time-series data being the time-series data generated by repeatedly transmitting and receiving ultrasound waves a plurality of times to the same position in a target examined region, using, as learning data, a combination of learning time-series data which are time-series data that have been generated based on a reflected wave from an examined region having the disease or blood flowing in the examined region by repeatedly transmitting and receiving ultrasound waves a plurality of times to and from the same position in the examined region and indicates a change in a signal over time and information indicating the disease in the examined region; and a notification unit that notifies a user of a result of the prediction by the disease prediction unit.

According to this configuration, upon inputting the target time-series data to the trained disease prediction learner, the disease prediction unit can predict the disease indicated by the target time-series data on the basis of the output of the disease prediction learner. As a result, the amount of calculation for predicting the disease indicated by the target time-series data is reduced as compared with the related art.

The disease prediction learner may be trained to output the possibility that the time-series data correspond to each of a plurality of diseases on the basis of the input time-series data, the disease prediction unit may input the target time-series data to the disease prediction learner to predict the possibility that the target time-series data corresponds to each of a plurality of diseases, and the notification unit may notify the user of the possibility that the target time-series data correspond to each of a plurality of diseases.

According to this configuration, the amount of calculation for predicting the possibility that the target time-series data corresponds to each of the plurality of diseases is reduced as compared with the related art, and the user can grasp the possibility that the target time-series data correspond to each of the plurality of diseases.

The notification unit may highlight and display, on a display unit, a disease to which the target time-series data are most likely to correspond among the plurality of diseases.

According to this configuration, the user can easily grasp the disease to which the target time-series data are most likely to correspond.

The disease prediction unit may identify the target examined region prior to the prediction of the disease indicated by the target time-series data, and further predict the disease indicated by the target time-series data on the basis of the identified region.

According to this configuration, the output accuracy of the disease prediction learner is improved, so that it is possible to improve the accuracy of the prediction of the disease indicated by the target time-series data.

The target examined region and the examined region may be pulsating regions, and the target time-series data and the learning time-series data may be time-series data corresponding to the same period in the pulsation cycle of the target examined region and the examined region.

According to this configuration, since the period of the learning time-series data and the period of the target time-series data in the pulsation period are the same, the output accuracy of the disease prediction learner is improved, and thus, it is possible to improve the accuracy of the prediction of the disease indicated by the target time-series data.

The ultrasound time-series data processing device disclosed in the present specification may further include a time-series data generation unit that generates the target time-series data, the disease prediction unit may predict the disease indicated by the target time-series data in real time in response to the generation of the target time-series data by the time-series data generation unit, and the notification unit may notify the user of the result of the prediction by the disease prediction unit in real time in response to the prediction of the disease by the disease prediction unit.

In the ultrasound time-series data processing device disclosed in the present specification, the amount of calculation for predicting the disease indicated by the target time-series data is reduced, and accordingly, the calculation time is also reduced. Therefore, in a case where the result of the prediction of the disease is notified to the user in real time as in the configuration, it is possible to reduce the delay in notification of the prediction result with respect to the time when the target time-series data are acquired.

An ultrasound time-series data processing program disclosed in the present specification causes a computer to function as:
a disease prediction unit that inputs target time-series data to a disease prediction learner and predicts a disease indicated by the target time-series data on the basis of an output of the disease prediction learner in response to the input, the disease prediction learner being trained to predict and output the disease indicated by the time-series data on the basis of the input time-series data, the target time-series data being the time-series data generated by repeatedly transmitting and receiving ultrasound waves a plurality of times to the same position in a target examined region, using, as learning data, a combination of learning time-series data which are time-series data that have been generated based on a reflected wave from an examined region having the disease or blood flowing in the examined region by repeatedly transmitting and receiving ultrasound waves a plurality of times to and from the same position in the examined region and indicates a change in a signal over time and information indicating the disease in the examined region; and a notification unit that notifies a user of a result of the prediction by the disease prediction unit.

According to the ultrasound time-series data processing device disclosed in the present specification, it is possible to reduce the amount of calculation for predicting a disease indicated by time-series data based on the time-series data obtained by repeatedly transmitting and receiving ultrasound waves a plurality of times to and from the same position in a subject.

### BRIEF DESCRIPTION OF DRAWINGS

Embodiment(s) of the present disclosure will be described based on the following figures, wherein:

FIG. 1 is a block diagram of an ultrasound diagnostic device according to the present embodiment;

FIG. 2 is a conceptual diagram illustrating a relationship between received beam data and received frame data;

FIG. 3 is a conceptual diagram illustrating a state of processing of training a disease prediction learner;

FIG. 4 is a conceptual diagram illustrating a state of prediction processing using the disease prediction learner;

FIG. 5 is a diagram illustrating a first example of a notification screen for notifying a prediction result of a disease prediction unit;

FIG. 6 is a diagram illustrating a second example of the notification screen for notifying the prediction result of the disease prediction unit; and

FIG. 7 is a flowchart illustrating a process performed by the ultrasound diagnostic device according to the present embodiment.

### DESCRIPTION OF EMBODIMENTS

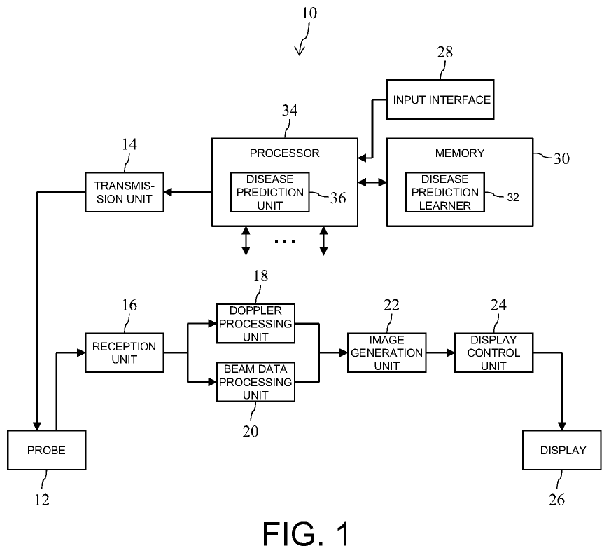

FIG. 1 is a block diagram of an ultrasound diagnostic device 10 serving as an ultrasound time-series data processing device according to the present embodiment. The ultrasound diagnostic device 10 is a medical device installed in a medical institution such as a hospital and is used for ultrasound inspection.

The ultrasound diagnostic device 10 is operable in a plurality of operation modes including a B mode, a Doppler mode, and an M mode. The B mode is a mode for generating and displaying a tomographic image (B-mode image) in which the amplitude intensity of a reflected wave from a scanned surface is converted into luminance on the basis of received frame data including a plurality of pieces of received beam data obtained by scanning with an ultrasound beam (transmission beam). The Doppler mode is a mode for generating and displaying a waveform (Doppler waveform) indicating the motion speed of tissue in an observation line, based on the difference between the frequency of a transmitted wave and the frequency of a reflected wave in the observation line set in a subject. The Doppler mode may include a continuous wave mode, a pulsed Doppler mode, a color Doppler mode, or a tissue Doppler mode. The M mode is a mode for generating and displaying an M-mode image representing tissue movement on the observation line set in the subject, based on received beam data corresponding to the observation line. The present embodiment particularly focuses on a case where the ultrasound diagnostic device 10 operates in the Doppler mode or the M mode.

A probe 12, which is an ultrasound probe, is a device that transmits an ultrasound wave and receives a reflected wave. Specifically, the probe 12 is brought into contact with the body surface of the subject, transmits an ultrasound wave toward the subject, and receives a wave reflected on tissue in the subject. A vibration element array including a plurality of vibration elements is provided in the probe 12. A transmission signal that is an electric signal is supplied from a transmission unit 14 to be described later to each of the vibration elements included in the vibration element array, whereby an ultrasound beam (transmission beam) is generated. In addition, each of the vibration elements included in the vibration element array receives a reflected wave from the subject, converts the reflected wave into a reception signal that is an electric signal, and transmits the reception signal to a reception unit 16 to be described later.

In order to transmit an ultrasound wave, the transmission unit 14 supplies a plurality of transmission signals to the probe 12 (specifically, the vibration element array) in parallel under the control of a processor 34 to be described later. As a result, the ultrasound wave is transmitted from the vibration element array.

In the Doppler mode or the M mode, the transmission unit 14 supplies the transmission signals to the probe 12 so that the probe 12 repeatedly transmits the transmission beam a plurality of times to the same position in an examined region of the subject determined by a user such as a doctor or a medical technician. In other words, the transmission unit 14 supplies the transmission signals to the probe 12 so that the probe 12 repeatedly transmits the transmission beam in a direction toward the same position in the examined region a plurality of times. In the B mode, the transmission unit 14 supplies the transmission signals to the probe 12 so that a scanning surface is electronically scanned with the transmission beam transmitted from the probe 12. Alternatively, time-division scanning may be performed to repeat transmission of the transmission beam to the same position determined by the user while the scanning surface is electronically scanned with the transmission beam.

At the time of receiving the reflected wave, the reception unit 16 receives a plurality of reception signals from the probe 12 (specifically, the vibration element array) in parallel. The reception unit 16 performs phasing addition (delay addition) on the plurality of reception signals, thereby generating received beam data.

In the Doppler mode or the M mode, the probe 12 repeats the transmission of the transmission beam to the same position in the examined region a plurality of times, so that the reception unit 16 receives a plurality of reflected waves from the examined region or blood flowing in the examined region, and generates a time-series received beam data sequence based on the plurality of reflected waves. In the B mode, the reception unit 16 configures the received frame data according to the plurality of pieces of received beam data arranged in the scanning direction.

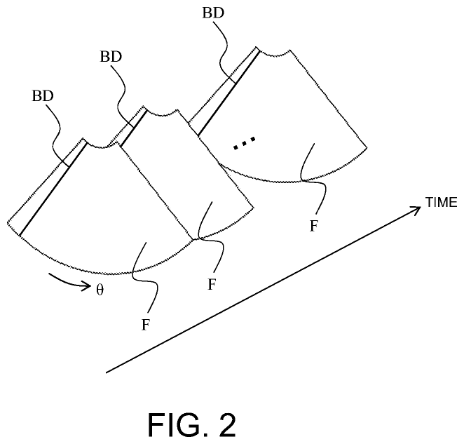

FIG. 2 is a conceptual diagram illustrating a relationship between the received beam data BD and the received frame data F. In the Doppler mode or the M mode, ultrasound waves are transmitted and received to and from a position (direction) designated by the user. As a result, a plurality of time-series pieces of received beam data DB (that is, received beam data sequence) are generated. The received beam data DB include information indicating the intensities and frequencies of the reflected waves from each depth. In the B mode, the scanning is performed with the transmission beam in the scanning direction θ, and the received frame data F is generated according to the plurality of pieces of received beam data arranged in the scanning direction θ.

In the Doppler mode, the received beam data sequence is transmitted to a Doppler processing unit 18. In the M mode, the received beam data sequence is transmitted to a beam data processing unit 20.

Returning to FIG. 1, in the Doppler mode the Doppler processing unit 18 generates Doppler data as time-series data indicating a change over time in the position of the examined region or a change over time in the velocity of the blood flowing in the examined region on the basis of the received beam data sequence from the reception unit 16. Specifically, the Doppler processing unit 18 generates the Doppler data by performing processing such as quadrature detection of multiplying received beam data by a reference frequency (transmission frequency) and extracting a Doppler shift through a low-pass filter, sample gate processing of extracting only a signal at a position of a sample volume (in the pulse Doppler mode), A/D conversion of a signal, and frequency analysis by a fast Fourier transform method (FFT method). The generated Doppler data are transmitted to an image generation unit 22 and the processor 34. In the Doppler mode, the reception unit 16 and the Doppler processing unit 18 correspond to a time-series data generation unit.

In the M mode, the beam data processing unit 20 performs various types of signal processing such as gain correction processing, logarithmic amplification processing, and filter processing on the received beam data sequence from the reception unit 16. The processed received beam data sequence is transmitted to the image generation unit 22 and the processor 34. In the present embodiment, in the M mode, the received beam data sequence processed by the beam data processing unit 20 corresponds to the time-series data indicating the change over time in the position of the examined region. In this case, the reception unit 16 and the beam data processing unit 20 correspond to a time-series data generation unit. Note that also in the B mode, the beam data processing unit 20 performs the above-described various types of signal processing on the received frame data from the reception unit 16.

The image generation unit 22 includes a digital scan converter, and includes a coordinate conversion function, a pixel interpolation function, a frame rate conversion function, and the like.

In the Doppler mode, the image generation unit 22 generates a Doppler waveform image on the basis of the Doppler data from the Doppler processing unit 18. The Doppler waveform is a waveform indicated on a two-dimensional plane of time and velocity, and indicates a change over time in the position of the examined region or a change over time in the velocity of the blood flowing in the examined region on the observation line corresponding to the received beam data sequence.

In the M mode, the image generation unit 22 generates an M-mode image on the basis of the received beam data sequence from the beam data processing unit 20. The M-mode image is a waveform indicated on a two-dimensional plane of time and depth, and indicates a change over time in the position of the examined region on the observation line corresponding to the received beam data sequence.

Note that, in the B-mode, the image generation unit 22 generates, on the basis of the received frame data from the beam data processing unit 20, a B-mode image in which the amplitude (intensity) of a reflected wave is represented by luminance.

A display control unit 24 causes a display 26 as a display unit including, for example, a liquid crystal panel or the like to display various images such as a Doppler waveform image, an M-mode image, or a B-mode image generated by the image generation unit 22. In addition, the display control unit 24 causes the display 26 to display a result of prediction by a disease prediction unit 36 to be described later.

Note that each of the transmission unit 14, the reception unit 16, the Doppler processing unit 18, the beam data processing unit 20, the image generation unit 22, and the display control unit 24 includes one or a plurality of processors, chips, electric circuits, and the like. Each of the units may be implemented by cooperation of hardware and software.

An input interface 28 includes, for example, one or more of a button, a track ball, a touch panel, and the like. The input interface 28 is for inputting a user's instruction to the ultrasound diagnostic device 10.

A memory 30 includes a hard disk drive (HDD), a solid state drive (SSD), an embedded multimedia card (eMMC), a read only memory (ROM), a random access memory (RAM), or the like. The memory 30 stores an ultrasound time-series data processing program for operating each unit of the ultrasound diagnostic device 10. Note that the ultrasound time-series data processing program can also be stored in a computer-readable non-transitory storage medium such as a universal serial bus (USB) memory or a CD-ROM. The ultrasound diagnostic device 10 or another computer can read and execute the ultrasound time-series data processing program from such a storage medium. Furthermore, as illustrated in FIG. 1, a disease prediction learner 32 is stored in the memory 30.

The disease prediction learner 32 includes, for example, a learning model such as a recurrent neural network (RNN), a long short term memory (LSTM) which is a type of RNN, a convolutional neural network (CNN), or a deep Q-network (DQN) using a deep reinforcement learning algorithm. The disease prediction learner 32 is trained to predict and output the disease indicated by the time-series data on the basis of the input time-series data, using, as learning data, a combination of learning time-series data which are time-series data generated based on a reflected wave from an examined region or blood flowing in the examined region by repeatedly transmitting and receiving ultrasound waves a plurality of times to and from the same position in the examined region having a disease, and information (label) indicating the disease in the examined region.

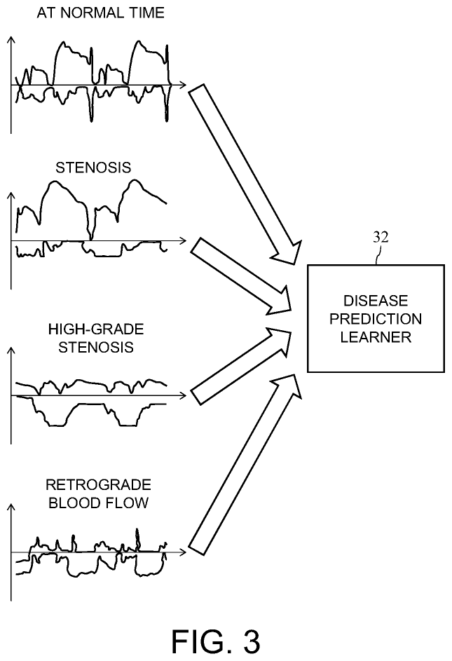

FIG. 3 is a conceptual diagram illustrating a state of processing of training the disease prediction learner 32. For example, learning time-series data obtained by transmitting and receiving ultrasound waves to and from an examined region having “stenosis” as a disease are input to the disease prediction learner 32. In this case, the disease prediction learner 32 predicts and outputs the disease indicated by the learning time-series data. An activation function such as a softmax function is provided in the last stage (output layer) of the disease prediction learner 32, and the disease prediction learner 32 outputs, as output data, the possibility (probability) that the learning time-series data may indicate a disease for each of a plurality of diseases. A computer that performs the training processing calculates an error between the output data and a label (in this case, “stenosis”) attached to the learning time-series data according to a predetermined loss function, and adjusts each parameter (for example, a weight or a bias of each neuron) of the disease prediction learner 32 so as to reduce the error. By repeating such training processing, the disease prediction learner 32 can output the possibility that the time-series data correspond to each of the plurality of diseases with high accuracy on the basis of the input time-series data.

The examined region to be a target of the learning time-series data may be a pulsating region. In this case, the learning time-series data may be data corresponding to a predetermined period in the pulsation cycle of the examined region. For example, an electrocardiographic waveform of the subject may be acquired from an electrocardiograph attached to the subject, and time-series data based on a received beam data sequence acquired in a period between R waves in the electrocardiographic waveform may be used as learning time-series data.

In the present embodiment, the learning time-series data are data (corresponding to the Doppler data or the received beam data sequence described above) before image conversion, but the learning time-series data may be a Doppler waveform image or an M-mode image (specifically, data obtained by quantifying the features of the Doppler waveform image or the M-mode image) generated on the basis of the Doppler data or the received beam data sequence.

Although FIG. 3 illustrates time-series data (normal time) indicating no disease as the learning data, the time-series data at the normal time is not necessarily included in the learning data. Furthermore, as the disease prediction learner 32, another learner may be prepared for each disease. In that case, each learner is trained to output a probability that the input time-series data indicate a corresponding disease.

In the present embodiment, the disease prediction learner 32 is trained by another computer other than the ultrasound diagnostic device 10, and the trained disease prediction learner 32 is stored in the memory 30. However, the processing of training the disease prediction learner 32 may be performed by the ultrasound diagnostic device 10 using the time-series data acquired by the ultrasound diagnostic device 10 as the learning time-series data. In this case, the processor 34 functions as a training processing unit that performs the processing of training the disease prediction learner 32.

Returning to FIG. 1, the processor 34 includes at least one of a general-purpose processing device (for example, a central processing unit (CPU) or the like) and a dedicated processing device (for example, a graphics processing unit (GPU), an application specific integrated circuit (ASIC), or a field-programmable gate array (FPGA), a programmable logic device, or the like). The processor 34 may be configured by cooperation of a plurality of processing devices present at physically separated positions, instead of one processing device. As illustrated in FIG. 1, the processor 34 functions as the disease prediction unit 36 according to the ultrasound time-series data processing program stored in the memory 30.

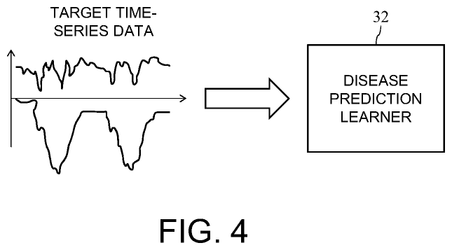

FIG. 4 is a conceptual diagram illustrating a state of prediction processing using the disease prediction learner 32. The disease prediction unit 36 inputs, to the trained disease prediction learner 32, time-series data (referred to as “target time-series data” in the present specification) obtained by repeatedly transmitting and receiving ultrasound waves a plurality of times to and from the same position in the target examined region. As described above, the target time-series data are generated by the reception unit 16 and the Doppler processing unit 18 (in the Doppler mode) or by the reception unit 16 and the beam data processing unit 20 (in the M mode). Furthermore, the target time-series data may be a Doppler waveform image or an M-mode image (specifically, data obtained by quantifying the features of the Doppler waveform image and the M-mode image) obtained on the basis of the Doppler data or the received beam data sequence.

The disease prediction learner 32 predicts the disease indicated by the target time-series data on the basis of the input target time-series data, and outputs output data indicating the prediction result. The disease prediction unit 36 predicts the disease indicated by the target time-series data on the basis of the output data of the disease prediction learner 32. As described above, in the present embodiment, the disease prediction learner 32 can output, as output data, the possibility indicated by the target time-series data for each of the plurality of diseases. For example, the disease prediction learner 32 outputs “0.12 (12%)” as the possibility that the target time-series data indicate a disease A, outputs “0.83 (83%)” as the possibility that the target time-series data indicate a disease B, outputs “0.06 (6%)” as the possibility that the target time-series data indicate a disease C, . . . , and outputs “0.03 (3%)” as the possibility that the target time-series data indicate a disease N. Based on such output data, the disease prediction unit 36 can predict the possibility that the target time-series data correspond to each of the plurality of diseases.

In a case where a plurality of disease prediction learners 32 are prepared for each disease, the disease prediction unit 36 sequentially transmits the target time-series data to the plurality of disease prediction learners 32, and predicts the possibility that the target time-series data correspond to each of the plurality of diseases on the basis of output data of each of the plurality of disease prediction learners 32.

As described above, in the present embodiment, when the target time-series data are input to the trained disease prediction learner 32, the disease prediction unit 36 can predict the disease indicated by the target time-series data on the basis of the output of the disease prediction learner 32. As a result, the amount of calculation for predicting the disease indicated by the target time-series data is reduced as compared with the related art.

When the target examined region and the examined region that is the target of the learning time-series data are pulsating regions, the target time-series data and the learning time-series data may be time-series data corresponding to the same period in the pulsation cycle of the target examined region and the examined region. For example, when the learning time-series data are time-series data based on a received beam data sequence acquired in a period between R waves in an electrocardiographic waveform, the disease prediction unit 36 may also set the target time-series data as time-series data based on the received beam data sequence acquired in the period between the R waves in the electrocardiographic waveform. Since the period of the learning time-series data and the period of the target time-series data in the pulsation cycle are the same, it is possible to improve the output accuracy of the disease prediction learner 32. That is, the accuracy of the prediction of the disease indicated by the target time-series data by the disease prediction unit 36 is improved.

The disease prediction unit 36 may identify the target examined region prior to the prediction of the disease indicated by the target time-series data, and further predict the disease indicated by the target time-series data on the basis of the identified region.

The target examined region can be identified by, for example, analyzing an image (Doppler waveform image or M-mode image) generated by the image generation unit 22 based on the target time-series data. In a case where the received frame data are acquired together with the time-series data by time-division scanning, the B-mode image generated by the image generation unit 22 on the basis of the received frame data may be analyzed to identify the target examined region. Alternatively, the disease prediction unit 36 may identify the target examined region on the basis of the user's input from the input interface 28. For example, the target examined region may be identified on the basis of a setting (for example, a preset selected by the user) for ultrasound diagnosis input from the input interface 28 by the user.

The disease prediction unit 36 inputs, to the disease prediction learner 32, a parameter indicating the target examined region together with the target time-series data. As a result, the disease prediction learner 32 can predict the disease indicated by the target time-series data while considering which region the target examined region is. For example, the disease prediction learner 32 can predict the disease indicated by the target time-series data after excluding a disease that cannot occur in the target examined region. As a result, the output accuracy of the disease prediction learner 32 can be improved; that is, the accuracy of the prediction of the disease indicated by the target time-series data by the disease prediction unit 36 is improved.

The display control unit 24 notifies the user of the prediction result of the disease prediction unit 36. That is, the display control unit 24 functions as a notification unit. Note that, in the present embodiment, the prediction result of the disease prediction unit 36 is displayed on the display 26 by the display control unit 24 as described below, but in addition to or instead of this, the prediction result of the disease prediction unit 36 may be notified to the user by voice output or the like.

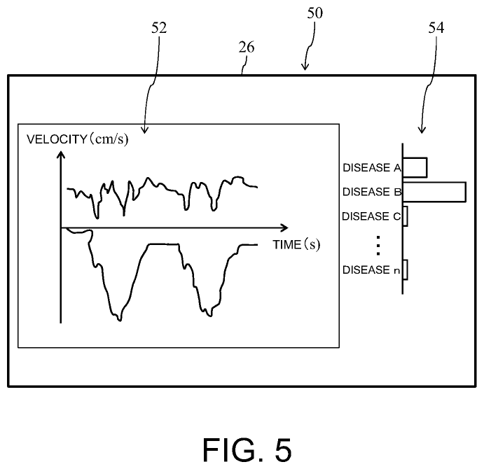

FIG. 5 is a diagram illustrating a first example of a notification screen 50 for notifying the prediction result of the disease prediction unit 36 displayed on the display 26. On the notification screen 50, an ultrasound image 52 generated on the basis of the target time-series data and a prediction result 54 of the disease prediction unit 36 are displayed. Note that the notification screen 50 is in the Doppler mode, and the Doppler waveform image is displayed as the ultrasound image 52. However, of course, in the M mode, the M mode image is displayed as the ultrasound image 52.

As described above, since the disease prediction unit 36 can predict the possibility that the target time-series data correspond to each of the plurality of diseases, the display control unit 24 may notify the user of the possibility that the target time-series data correspond to each of the plurality of diseases. In the prediction result 54 on the notification screen 50, the display control unit 24 displays a plurality of disease names (“disease A”, “disease B”, “disease C”, . . . , “disease N”) and displays the possibility that the target time-series data correspond to each of the diseases in the form of a graph.

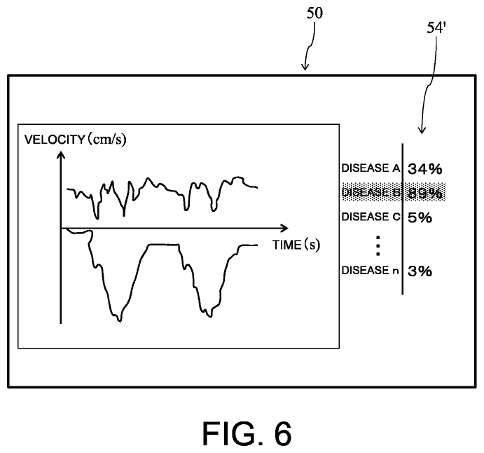

FIG. 6 is a diagram illustrating a second example of the notification screen 50. As illustrated in the notification screen 50 in FIG. 6, the display control unit 24 may highlight and display, in a prediction result 54′, a disease that is most likely to correspond to the target time-series data among the plurality of diseases for which the disease prediction unit 36 has predicted the possibility that the target time-series data correspond to each of the diseases. In the example illustrated in FIG. 6, since the target time-series data are most likely to correspond to “disease B” among the plurality of diseases, “disease B” is highlighted in the prediction result 54′.

As an aspect of the highlighted display, various aspects are conceivable. For example, a disease to be highlighted may be displayed in a color or font different from that of the other diseases. In addition, a disease to be highlighted may be displayed with a marker, shading, or the like. In addition, the plurality of diseases may be arranged in descending order of possibility that the target time-series data correspond thereto. In addition, only a disease to be highlighted (a disease to which the target time-series data are most likely to correspond) may be displayed.

By highlighting the disease to which the target time-series data are most likely to correspond among the plurality of diseases, the user can easily grasp the disease to which the target time-series data are likely to correspond.

As described above, in the present embodiment, the amount of calculation for predicting the disease indicated by the target time-series data is reduced as compared with the related art. That is, the prediction of the disease indicated by the target time-series data can be performed at a higher speed. Therefore, the disease prediction unit 36 may predict the disease indicated by the target time-series data in real time in response to the generation of the target time-series data, and the display control unit 24 may notify the user of the result of the prediction by the disease prediction unit 36 in real time in response to the prediction of the disease indicated by the target time-series data by the disease prediction unit 36. According to the present embodiment, even if such real-time processing is performed, it is possible to smoothly notify the user of the prediction result of the disease prediction unit 36 without causing delay or the like.

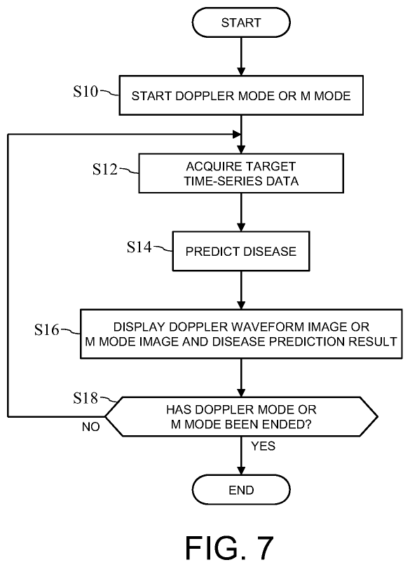

Hereinafter, a process performed by the ultrasound diagnostic device 10 according to the present embodiment will be described with reference to a flowchart illustrated in FIG. 7.

In step S10, the ultrasound diagnostic device 10 starts the Doppler mode or the M mode in response to a user's instruction from the input interface 28.

In step S12, in the Doppler mode, the Doppler processing unit 18 generates Doppler data as the target time-series data on the basis of a received beam data sequence from the reception unit 16. In the M mode, the beam data processing unit 20 generates a received beam data sequence subjected to various types of signal processing as the target time-series data.

In step S14, the disease prediction unit 36 inputs the target time-series data (Doppler data or received beam data sequence) generated in step S12 to the trained disease prediction learner 32. The disease prediction unit 36 predicts the disease indicated by the target time-series data on the basis of the output data of the disease prediction learner 32 for the target time-series data.

In step S16, the display control unit 24 causes the display 26 to display a Doppler waveform image based on the Doppler data generated in step S12 or an M-mode image based on the received beam data sequence generated in step S12, and the result of the prediction by the disease prediction unit 36 in step S14. As a result, the result of the prediction by the disease prediction unit 36 is notified to the user.

In step S18, the processor 34 determines whether or not the Doppler mode or the M mode is ended according to the user's instruction. When the Doppler mode or the M mode is continued, the process returns to step S12, and the processing from steps S12 to S18 is repeated. When the Doppler mode or the M mode is ended, the process is ended.

Although the ultrasound time-series data processing device according to the present disclosure has been described above, the ultrasound time-series data processing device according to the present disclosure is not limited to the above-described embodiment, and various modifications can be made without departing from the gist thereof.

For example, in the present embodiment, the ultrasound time-series data processing device is the ultrasound diagnostic device 10, but the ultrasound time-series data processing device is not limited to the ultrasound diagnostic device 10, and may be another computer. In this case, the trained disease prediction learner 32 is stored in a memory accessible from a computer as the ultrasound time-series data processing device, and a processor of the computer functions as the disease prediction unit 36 and the display control unit 24.

1. An ultrasound time-series data processing device comprising:
a disease prediction unit that inputs target time-series data to a disease prediction learner and predicts a disease indicated by the target time-series data on the basis of an output of the disease prediction learner in response to the input, the disease prediction learner being trained to predict and output the disease indicated by the time-series data on the basis of the input time-series data, the target time-series data being the time-series data generated by repeatedly transmitting and receiving ultrasound waves a plurality of times to the same position in a target examined region, using, as learning data, a combination of learning time-series data which are time-series data that have been generated based on a reflected wave from an examined region having the disease or blood flowing in the examined region by repeatedly transmitting and receiving ultrasound waves a plurality of times to and from the same position in the examined region and indicate a change in a signal over time and information indicating the disease included in the examined region; and
a notification unit that notifies a user of a result of the prediction by the disease prediction unit.

2. The ultrasound time-series data processing device according to claim 1, wherein
the disease prediction learner is trained to output a possibility that the time-series data correspond to each of a plurality of diseases on the basis of the input time-series data,
the disease prediction unit inputs the target time-series data to the disease prediction learner to predict the possibility that the target time-series data correspond to each of the plurality of diseases, and
the notification unit notifies the user of the possibility that the target time-series data correspond to each of the plurality of diseases.

3. The ultrasound time-series data processing device according to claim 2, wherein the notification unit highlights and displays, on a display unit, a disease to which the target time-series data are most likely to correspond among the plurality of diseases.

4. The ultrasound time-series data processing device according to claim 1, wherein the disease prediction unit identifies the target examined region prior to the prediction of the disease indicated by the target time-series data, and further predicts the disease indicated by the target time-series data on the basis of the identified region.

5. The ultrasound time-series data processing device according to claim 1, wherein
the target examined region and the examined region are pulsating regions, and
the target time-series data and the learning time-series data are time-series data corresponding to the same period in a pulsation cycle of the target examined region and the examined region.

6. The ultrasound time-series data processing device according to claim 1, further comprising
a time-series data generation unit that generates the target time-series data, wherein
the disease prediction unit predicts the disease indicated by the target time-series data in real time in response to the generation of the target time-series data by the time-series data generation unit, and
the notification unit notifies the user of the result of the prediction by the disease prediction unit in real time in response to the prediction of the disease indicated by the target time-series data by the disease prediction unit.

7. A computer-readable non-transitory storage medium storing a computer-executable command, the command causing a computer to execute:
a disease prediction step of inputting target time-series data to a disease prediction learner and predicting a disease indicated by the target time-series data on the basis of an output of the disease prediction learner in response to the input, the disease prediction learner being trained to predict and output the disease indicated by the time-series data on the basis of the input time-series data, the target time-series data being the time-series data generated by repeatedly transmitting and receiving ultrasound waves a plurality of times to the same position in a target examined region, using, as learning data, a combination of learning time-series data which are time-series data that have been generated based on a reflected wave from an examined region having the disease or blood flowing in the examined region by repeatedly transmitting and receiving ultrasound waves a plurality of times to and from the same position in the examined region and indicate a change in a signal over time and information indicating the disease in the examined region; and
a notification step of notifying a user of a result of the prediction by the disease prediction unit.
